SEQUENCE LISTING (11 sequences)

SEQ ID NO: 1 - DNA, 10 bp, artificial sequence
  synthethic primers - operon / design
caggcccttc

SEQ ID NO: 2 - DNA, 10 bp, artificial sequence
  Primers-Opreon/Design
tgccgcgctg

SEQ ID NO: 3 - DNA, 10 bp, artificial sequence
  Primers-Opreon/Design
agtcagccac

SEQ ID NO: 4 - DNA, 10 bp, artificial sequence
  Primers-Opreon/Design
gaaacgggtg

SEQ ID NO: 5 - DNA, 10 bp, artificial sequence
  Primers-Opreon/Design
gtgacgtagg

SEQ ID NO: 6 - DNA, 10 bp, artificial sequence
  Primers-Opreon/Design
gggtaacgcc

SEQ ID NO: 7 - DNA, 10 bp, artificial sequence
  SEQ ID NO 8
gtgatcgcag

SEQ ID NO: 8 - DNA, 10 bp, artificial sequence
  Primers-Opreon/Design
taggatcaga

SEQ ID NO: 9 - DNA, 10 bp, artificial sequence
  Primers-Opreon/Design
aaggatcaga

SEQ ID NO: 10 - DNA, 10 bp, artificial sequence
  Primers-Opreon/Design
cacatgcttc

SEQ ID NO: 11 - DNA, 10 bp, artificial sequence
  Primers-Opreon/Design
aggtcactga

(19) United States
(12) Patent Application Publication
(10) Pub. No.: US 2015/0176028 A1
(43) Pub. Date: Jun. 25, 2015

(54) XYLOSE FERMENTING YEAST CONSTRUCTED USING A MODIFIED GENOME SHUFFLING METHOD

(71) Applicant: NGEE ANN POLYTECHNIC, Singapore (SG)

(72) Inventors: Anli Geng, Singapore (SG); Wei Zhang, Singapore (SG)

(21) Appl. No.: 14/403,997

(22) PCT Filed: May 27, 2013
(86) PCT No.: PCT/SG2013/000214

§ 371 Date: Nov. 25, 2014

(30) Foreign Application Priority Data
May 25, 2012 (SG) 201203879-0

(51) Int. Cl. C12N 15/87; C12P 7/10

(52) U.S. Cl. CPC C12N 15/87; C12P 7/10

(57) ABSTRACT

Disclosed is a method of providing a recombinant microorganism. The method comprises the steps of: (a) providing a hybrid microorganism comprising DNA from a host microorganism and a donor microorganism; and (b) fusing DNA extracted from a second microorganism into the hybrid microorganism to form the recombinant microorganism. Also disclosed are recombinant yeasts produced by the method of the present disclosure and a method of fermenting sugar using the recombinant yeast produced by the method of the present disclosure.

This application claims the benefit of priority of Singapore Patent Application No. 201203879-0, filed 25 May 2012, the contents of it being hereby incorporated by reference in its entirety for all purposes.

### TECHNICAL FIELD

The present invention generally relates to the field of yeast cloning and expression. In particular, the present invention relates to a method of producing recombinant yeast strain and use of the recombinant yeast strain.

### BACKGROUND

In recent years, there has been a growing interest in the utilization of renewable resources for the production of bioethanol, which has been deemed as the cleanest liquid fuel alternative to fossil fuels. Apart from starch crops and sugarcane, lignocellulosic biomass, such as wood waste and agricultural waste, has been considered as the feedstock with the most potential for bioethanol production as it is the most abundant source of sugars and does not compete with the food resource.

Xylose is the second most abundant sugar present in lignocellulosic biomass after glucose. The efficient fermentation of xylose is required to develop economically viable processes for the production of bioethanol from lignocellulosic biomass.

Saccharomyces cerevisiae is regarded as an industrial working horse for ethanol production because it can produce ethanol in high titre using hexose sugars and have high ethanol tolerance. However, it cannot ferment xylose.

Pichia stipitis is one of the most efficient naturally occurring xylose-fermenting yeasts and it can convert xylose to ethanol in high yield. However, it has low ethanol and sugar tolerance. This feature of P. stipitis has limited its use as an industrial strain for large-scale bioethanol production from lignocellulosic biomass.

The primary desired traits of an industrial strain required for fermenting lignocellulosic hydrolysate are efficient utilization of hexoses and pentoses, high fermentation rates, and high ethanol production, high tolerance to ethanol, sugars and fermentation inhibitors.

While rational metabolic engineering has been effective in improving phenotypes of S. cerevisiae strains for xylose fermentation, it typically involves the constitutive expression of multiple genes followed by mutagenesis and post-evolutionary engineering. It is therefore tedious, labour intensive and time-consuming.

On the other hand, the whole genome engineering approach, such as genome shuffling, offers the advantage of simultaneous changes at different positions throughout the entire genome without the necessity for genome sequence data or network information. It therefore has advanced the field of constructing phenotypes at a more rapid pace as compared with the conventional tools.

Considering the complexity of pathway design for rational metabolic engineering, genome shuffling uses recursive genetic recombination analogous to DNA shuffling. This strategy has been successfully applied in rapid strain improvement of both prokaryotic and eukaryotic cells. However, this method largely depends on the efficiency of the traditional protoplast fusion techniques. Although protoplast fusion has been regarded as a traditional and effective way to accelerate strain evolution and has been applied in many studies, the technique has the disadvantages of low efficiency of fusion induced by polyethylene glycol (PEG), being labour intensive and time-consuming due to protoplast preparation and fusant regeneration, and fusant instability.

In view of the problems mentioned above, there is a need to provide a method of providing a recombinant microorganism that ameliorates at least one of the disadvantages mentioned above. There is a need to provide a method of producing recombinant yeast strains that is rapid and reliable. In particular, there is a need to provide recombinant yeast strains with enhanced xylose-fermentation capability and which have enhanced ethanol tolerance.

### SUMMARY

In one aspect, there is provided a method of providing a recombinant microorganism, said method may comprising the steps of (a) providing a hybrid microorganism comprising DNA from a host microorganism and a donor microorganism; and (b) fusing DNA extracted from a second microorganism into the hybrid microorganism to form the recombinant microorganism.

In another aspect, there is provided a recombinant yeast strain produced according to the method as disclosed herein.

In yet another aspect, there is provided a method of fermenting sugar with the recombinant yeast strain as disclosed herein to produce ethanol.

In yet another aspect, there is provided a method of fermenting a sugar mixture having a high sugar content with the recombinant yeast strain as disclosed herein to produce ethanol wherein the total sugar content is greater than 50 grams per liter.

### BRIEF DESCRIPTION OF THE DRAWINGS

The invention will be better understood with reference to the detailed description when considered in conjunction with the non-limiting examples and the accompanying drawings. The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a graph showing the fermentation profile of P. stipitis, F1-8 and ScF2. Recombinant strains F1-8 and ScF2, and parent strain, P. stipitis, were evaluated in 150 mL shaking flasks filled with 50 mL of fermentation medium containing 120 g/L xylose. FIG. 1 illustrates recombinant strain ScF2 having improved ethanol production rate and ethanol titre as compared to both P. stipitis and F1-8.

FIG. 2 is a photo of gel electrophoresis analysis of genetic variation of yeasts produced by Random Amplified Polymorphic DNA (RAPD) reactions performed using random primer kit. (a) RAPD profiles of S. cerevisiae, P. stipitis, F1-8 and ScF2; Lad is the DNA ladder. (b) Observance of genetic stability of ScF2; Lanes 1-6 are S. cerevisiae, P. stipitis, 1 kb DNA ladder, ScF2 (February 2011), ScF2 (June 2011), and ScF2 (November 2011), respectively. FIG. 2 illustrates the differences in RAPD profiles of parent and ScF2 strains (FIG. 2(a)), and the consistency in RAPD profiles in recombinant yeast stored at different time points (FIG. 2(b)).

FIG. 3 depicts graphs showing the results of time course studies of cell growth, xylose consumption and ethanol production by P. stipitis and ScF2 in high initial xylose concentrations. Xylose fermentation study was conducted in high initial xylose concentrations (i.e. 100 (a), 150 (b), 200 (c) and 250 (d) g/L) at 30° C. and 100 rpm. Filled symbols, P. stipitis; empty symbols, ScF2. FIG. 3 illustrates higher xylose tolerance and improved ethanol titre in recombinant ScF2 as compared to the parent P. stipitis.

FIG. 4 is a graph showing the result of time course study of xylitol production by P. stipitis and ScF2. 150 g/L xylose was fermented in fermentation medium at 30° C. FIG. 4 shows higher xylitol production rate in recombinant yeast ScF2 as compared to the parent P. stipitis.

FIG. 5 depicts graphs showing the result of time course study of fermentation of glucose, xylose, and a mixture thereof by P. stipitis, S. cerevisiae and ScF2. (a) and (b): filled symbols, glucose or xylose; empty symbols, ethanol. (c): filled symbols, glucose; empty symbols, xylose; crossed empty symbols, ethanol. The fermentation of glucose, xylose, and a mixture thereof were investigated independently under batch cultivation conditions. Initial total sugar concentration of 100 g/L was used. Glucose level was measured at various time points. FIG. 5 illustrates recombinant yeast ScF2 to exhibit higher rates of both xylose consumption and ethanol production than P. stipitis.

FIG. 6 is a graph showing fermentation profile of ScF2 precultured in high-concentration glucose or xylose. Xylose fermentation performance was studied in yeast peptone medium containing 10 g/L yeast extract, 20 g/L peptone and 150 g/L glucose or xylose. Cells were harvested and inoculated into fresh fermentation medium containing 150 g/L xylose at an initial OD₆₀₀ of 3.0. Filled symbols, ScF2 precultured on glucose; Empty symbols: ScF2 precultured on xylose. FIG. 6 shows that ScF2 presented higher xylose consumption rate and ethanol productivity compared to P. stipitis despite the pre-culturing conditions.

FIG. 7 is a graph showing high density xylose fermentation by ScF2. ScF2 was prepared in yeast peptone medium containing 10 g/L yeast extract, 20 g/L peptone, and 150 g/L glucose or xylose. Cells were harvested and inoculated into fresh fermentation medium containing 150 g/L xylose at an initial OD₆₀₀ of 40.0. Filled symbols, ScF2; Empty symbols, Pichia stipitis. Square, xylose; triangle, ethanol; circle, cell biomass. FIG. 7 shows significantly higher ethanol productivity by recombinant yeast ScF2 at high cell density and high ethanol titer.

FIG. 8 depicts graphs showing simultaneous saccharification and fermentation (SSF) of oil palm empty fruit bunch (OPEFB). (a) displays results of hydrolysis of OPEFB at 30° C. (b) and (c) show simultaneous saccharification and fermentation resulting in cellulosic ethanol production at 30° C. using P. stipitis and ScF2, respectively. Thus, FIG. 8 demonstrates both ethanol titre and ethanol productivity improvement in ScF2.

### BRIEF DESCRIPTION OF THE TABLES

Table 1 shows the fermentation performance of first round hybrid yeasts in YNBX broth containing 150 g/L xylose.

Table 2 is an exemplary list of primers used for random amplified polymorphic DNA used in the present disclosure.

Table 3 illustrates sugar utilization by ScF2 and its parental strains.

Table 4 lists xylose fermentation parameters with ScF2 inoculum pre-cultured in 150 g/L glucose or xylose.

### DISCLOSURE OF OPTIONAL EMBODIMENTS

Disclosed is an example of a rapid and reliable modified genome shuffling method to construct a recombinant yeast strain with improved xylose fermentation that does not require a protoplast fusion step. In one aspect, there is provided a method of providing a recombinant microorganism. The method may comprise the step of: (a) providing a hybrid microorganism comprising DNA from a host microorganism and a donor microorganism; and (b) fusing DNA extracted from a second microorganism into the hybrid microorganism to form the recombinant microorganism. In one embodiment, the method as disclosed herein does not involve yeast sexual and asexual reproduction.

As used herein, “microorganism” in the present disclosure includes, but is not limited to, bacteria, fungi and yeast. In one embodiment, the microorganism may be a yeast cell. In one embodiment, the microorganism may be capable of fermenting xylose. In one embodiment, the microorganism may be tolerant to ethanol.

As used herein, the term “recombinant” refers to a microorganism that does not occur naturally in nature. For example, the “recombinant microorganism” may express genes that are not found in identical form within the native (i.e., non-recombinant) form of the cell and/or express native genes that are otherwise abnormally over-expressed, under-expressed, and/or not expressed at all due to deliberate human intervention. A recombinant microorganism contains at least one recombinant polynucleotide or polypeptide. A nucleic acid construct, nucleic acid (e.g., a polynucleotide), polypeptide, or host cell is referred to herein as “recombinant” when it is non-naturally occurring, artificial or engineered. “Recombination”, “recombining”, and generating a “recombined” DNA generally encompass the assembly of at least two nucleic acid fragments. In one embodiment, the recombinant microorganism and recombinant nucleic acids remain functional, i.e., retain their activity or exhibit an enhanced activity. In the present disclosure, the term “recombinant microorganism” may specifically refer to the final microorganism resulting from the method as described herein. In one embodiment, the recombinant microorganism may be a recombinant yeast strain produced by the method as described herein. In one embodiment, no yeast sexual and asexual production is involved in the formation of the recombinant microorganism.

As used herein, the term “hybrid microorganism” refers to microorganism that does not occur naturally in nature, that express genomes that are not found in identical form within the native form of the microorganism and/or express native genomes that are otherwise abnormally over-expressed, under-expressed, and/or not expressed at all due to deliberate human intervention. In particular, a “hybrid microorganism” may refer to a microorganism having genomes of two different parent microorganisms. In one embodiment, a “hybrid microorganism” comprises DNA from a host microorganism and a donor microorganism. In one embodiment, the hybrid microorganism may be produced by transferring the whole genome of a donor microorganism to a host microorganism by whole genome transformation. In one embodiment, the hybrid microorganism may be produced by transferring the whole genome of P. stipitis to S. cerevisae by electroporation. In one embodiment, no yeast sexual and asexual production is involved in the formation of the hybrid microorganism. In one embodiment, a hybrid microorganism exhibits an enhanced activity as compared to its host microorganism and/or donor microorganism individually. In one embodiment, the hybrid microorganism may be obtained by screening a plurality of hybrid microorganisms for capability to ferment xylose. In one embodiment, the plurality of hybrid microorganisms may be provided by mixing a suspension of the host microorganism with extracted DNA from the donor microorganism to achieve transfection of the extracted DNA into the host microorganism. In one embodiment, the hybrid microorganism is a hybrid yeast cell.

As used herein, “host microorganism” refers to microorganism that contains a polynucleotide, gene, promoter or polypeptide endogenous to the microorganism that is not removed from the microorganism. In one embodiment, the “host microorganism” may be subjected to laboratory manipulation that introduces heterologous genome from a donor microorganism. In one embodiment, the “host microorganism” may be a yeast cell that is capable of fermenting xylose. In one embodiment, the “host microorganism” may be a yeast cell that is tolerant to ethanol. The host yeast cell may include, but is not limited to, a yeast cell from the genus Brettanomyces, Candida, Citeromyces, Cyniclomyces, Debaryomyces, Issatchenkia, Kazachstania, Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, and Zygotorulaspora. In one embodiment, the host yeast cell may be of the genus Saccharomyces. In one embodiment, the host yeast cell may include, but is not limited to, a yeast cell of the species Saccharomyces cerevisiae, Saccharomyces pastorianus, Saccharomyces boulardii, Saccharomyces eubayanus, Saccharomyces bayanus, Saccharomyces bailii, and Saccharomyces florentinus. In one embodiment, the host cell may be of the species Saccharomyces cerevisiae.

As used herein, the term “donor microorganism” refers to any microorganism that has its polynucleotide, gene, promoter or polypeptide removed from the microorganism and then reintroduced into a host microorganism. In one embodiment, the donor microorganism may be capable of fermenting xylose. In one embodiment, the “donor microorganism” may be tolerant to ethanol. In one embodiment, the “donor microorganism” may belong to a different taxonomic family as the “host microorganism”. In one embodiment, the “donor microorganism” may belong to the same taxonomic family as the “host microorganism”. In one embodiment, the donor microorganism is a yeast cell. In one embodiment, the donor yeast cell belongs to the family of Saccharomycetaceae. In one embodiment, the donor yeast cell may include, but is not limited to, a yeast cell from the genus Brettanomyces, Candida, Citeromyces, Cyniclomyces, Debaryomyces, Issatchenkia, Kazachstania, Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, and Zygotorulaspora. In one embodiment, the donor yeast cell is of the genus of Pichia. In one embodiment, the donor yeast cell may be a species, including, but is not limited to, Pichia pastoris, Pichia guilliermondii, Pichia membranifaciens, Pichia heedii, Pichia stipitis, and Pichia subpelliculosa. In one embodiment, the donor yeast cell is of the species Pichia stipitis.

In one embodiment, the host microorganism and/or donor microorganism may be yeast cells. In one embodiment, the host and for donor yeast cell may be capable of fermenting xylose. The host and/or donor yeast cell may be tolerant to ethanol. In one embodiment, the host and/or donor yeast cells may belong to different taxonomic families. In one embodiment, the host and/or donor yeast cells may belong to the same taxonomic family. In one embodiment, the host and/or donor yeast cells belong to the family of Saccharomycetaceae.

As used herein, the term “fusing” refers to the combination of DNA extracted from at least a first microorganism into a second microorganism. The combination may be random. Fusing may be brought into contact under conditions stimulating fusing, such as transfection and/pr transformation. The first and/or second microorganism may be a haploid microorganism, a diploid microorganism and/or a hybrid microorganism derived from two or more microorganism that contains DNA sequences to be recombined or already recombined DNA sequences. In one embodiment, the second microorganism may be of the same or different species as the first microorganism. In another embodiment, the second microorganism is of the same species as the first microorganism. The term “fusing” may not include sexual and asexual reproduction of the first and second microorganism.

In one embodiment, the method of providing a recombinant microorganism as disclosed herein may further comprise the steps of: (c) screening the recombinant microorganism and selecting a recombinant strain expressing two or more desired traits; and (b) fusing DNA extracted from a microorganism expressing at least one of the desired traits into said selected recombinant strain to obtain further recombinant microorganism. For example, where there are two desired traits, the DNA is extracted from a microorganism expressing both of the desired traits into the selected recombinant strain to obtain a further recombinant microorganism. In one example, the method may further comprise repeating steps (c) and (d) until the desired traits are expressed in the recombinant microorganism. In one embodiment, the host or donor yeast cell may be a yeast cell. Exemplary desired traits include, but are not limited to, enhanced xylose-fermentation capability and have enhanced ethanol tolerance.

As used herein, the term “extract,” “extracted,” or grammatical variants thereof, refers to the removal, isolation or purification of a molecule (e.g., an isolated nucleic acid, polypeptide, etc.) or other component from at least one other component or microorganism with which it is naturally associated.

In one embodiment, when the hybrid microorganism is a hybrid yeast cell, the desired hybrid microorganism may be obtained by screening a plurality of hybrid yeast cell lines for capability to ferment xylose. In one embodiment, the plurality of hybrid yeast cell lines may be provided by mixing a suspension of a host yeast cell with extracted DNA from a donor yeast cell to achieve transfection of the extracted DNA into the host yeast cell.

In one embodiment, the transfection as used in the present disclosure may be carried out using a transfection method including, but not limited to, chemical based transfection, non-chemical based transfection, particle-based transfection and viral methods.

In embodiments where transfection is carried out using chemical based transfection, the method may be carried out using, but is not limited to, calcium phosphate or dendrimers or liposomes or cationic polymers.

In embodiments where transfection is carried out using non-chemical transfection, the method may be carried out by a method including, but is not limited to, electroporation or sono-poration or optical transfection or gene electrotransfer or hydrodynamic delivery.

In embodiments where transfection is carried out using particle-based transfection, the method may be carried out using, but is not limited to, a gene gun or magnetofection or impalefection.

Also disclosed is a method of fermenting sugar with the recombinant microorganism. In one embodiment, the recombinant microorganism may be a recombinant yeast strain. As used herein, the terms “ferment”, “fermenting” and “fermentation” refer to a biochemical process in which a carbon source (e.g., a sugar) is broken down to produce at least one fermentation product, including but not limited to such products as alcohols (e.g., ethanol, butanol, etc.), fatty alcohols (e.g., C8-C20 fatty alcohols), acids (e.g., lactic acid, 3-hydroxypropionic acid, acrylic acid, acetic acid, succinic acid, citric acid, malic acid, fumaric acid, amino acids, etc.), 1,3-propane diol, ethylene glycol, glycerol, terpenes, and antimicrobials (e.g., β-lactams such as cephalosporin), etc. In some embodiments in which ethanol is produced by fermentation, other products, including but not limited to lactate, acetic acid, hydrogen and carbon dioxide may also be produced.

Thus, also disclosed is a method of fermenting sugar with recombinant yeast as described herein to produce ethanol. In one embodiment, the sugar may include, but is not limited to, hexoses, pentoses, disaccharides and mixtures thereof. The hexoses may include, but are not limited to, glucose, fructose, galactose, mannose, rhamnose and mixtures thereof. The pentoses may include, but are not limited to, xylose, L-arabinose, D-arabinose, ribose and mixtures thereof. The disaccharides may include, but are not limited to, sucrose, lactose, cellobiose, maltose and mixtures thereof. In one embodiment, the sugar is xylose. In one embodiment, the sugar mixture may be composed essentially of xylose.

In one embodiment, the method as disclosed herein may produce a recombinant yeast that may be used to convert lignocellulosic biomass, such as agricultural waste, forest and wood waste to ethanol and then to other value-added products such as aviation fuels, acetaldehyde, butanol, ethylene, polyethylene, and the like.

Also disclosed is a method of fermenting a sugar mixture having a high sugar content with the recombinant microorganism as described herein to produce ethanol. In one embodiment, the recombinant microorganism may be a recombinant yeast strain as described herein. In one embodiment, the total sugar content may be greater than 10 grams per liter, 20 grams per liter, 30 grams per liter, 40 grams per liter, 50 grams per liter, 60 grams per liter, 70 grams per liter, 80 grams per liter, 90 grams per liter, 100 grams per liter, 110 grams per liter, 120 grams per liter, 130 grams per liter, 140 grams per liter, 150 grams per liter, 160 grams per liter, 170 grams per liter, 180 grams per liter, 190 grams per liter, 200 grams per liter, 210 grams per liter, 220 grams per liter, 230 grams per liter, 240 grams per liter, 250 grams per liter, 260 grams per liter, 270 grams per liter, 280 grams per liter, 290 grams per liter, 300 grams per liter or more. In one embodiment, the total sugar content may be greater than 50 grams per liter. In one embodiment, the total sugar content may be from about 10, 20, 30, 40, 50, 60, 70, 80, 90, 100, 110, 120, 130, 140, 150, 160, 170, 180, 190, 200, 210, 220, 230, 240, 250, 260, 270, 280, 290 grams per liter to about 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400 grams per liter. In one embodiment, the total sugar content may be about 10 to about 400 grams per liter, about 50 to about 350 grams per liter, about 100 to about 300 grams per liter, about 100 to about 250 grams per liter or about 150 to about 200 grams per liter.

In one embodiment, a method of providing a recombinant yeast is provided as illustrated in the Experimental Section of the present disclosure. This method was based on the recombination of whole genomes from different yeast strains in vivo. Genomic DNA of one parent strain was extracted and transferred into the other parental strain to allow the recombination of the two genomes to form a hybrid strain. Potential recombinant strains with the desired features were selected on the pre-designed screening plates. Their fermentation performance were then evaluated and compared.

In one embodiment, the method of providing a recombinant yeast of the present disclosure involves two steps of genome shuffling. In one embodiment, the method as disclosed herein may use a haploid yeast and a diploid yeast. In the first step, the whole genome of a haploid yeast may be extracted and transferred into a diploid yeast by electroporation to thereby form a hybrid yeast. A desired hybrid yeast would have the best ethanol production performance as tested using methods known in the art. In the second step, the whole genome of a diploid yeast, which may be the same as the diploid yeast used in the first step, may be transferred into the hybrid yeast by electroporation, thereby forming the recombinant yeast. A desired recombinant yeast is then selected by observing xylose fermentation capability and ethanol tolerance level. In one embodiment, the desired trait may include, but is not limit to, high concentration ethanol (product) tolerance, high concentration inhibitor resistance, high concentration substrate tolerance, high or low temperature tolerance, high or low pH tolerance, broad pH stability, high temperature stability, broad substrates utilization capability, high product titer and high productivity.

In one embodiment, the method as described herein does not involve protoplast fusion techniques and yeast sexual or asexual reproduction. The method as described herein may involve recursive recombination of a donor genome with a host genome through direct genome isolation and transformation. In one embodiment, the method provides the rapid construction of a recombinant yeast strain with enhanced xylose-fermentation using a modified genome shuffling method, which involves the recursive recombination of P. stipitis genome with that of S. cerevisiae through direct genome isolation and transformation.

In one embodiment, there is provided a recombinant yeast ScF2 as used in the Experimental Section.

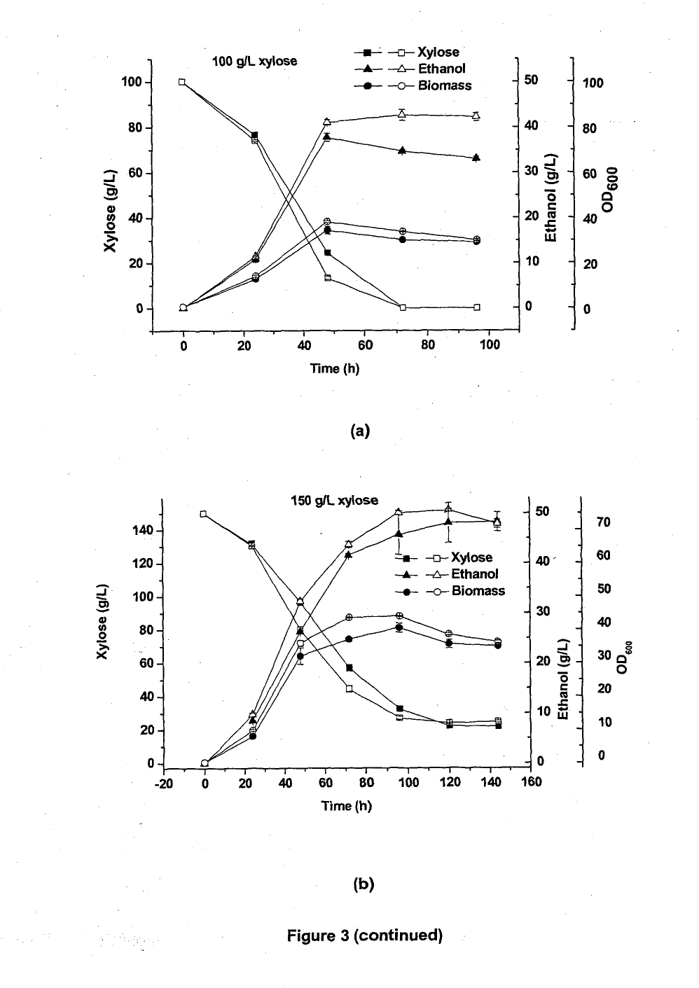

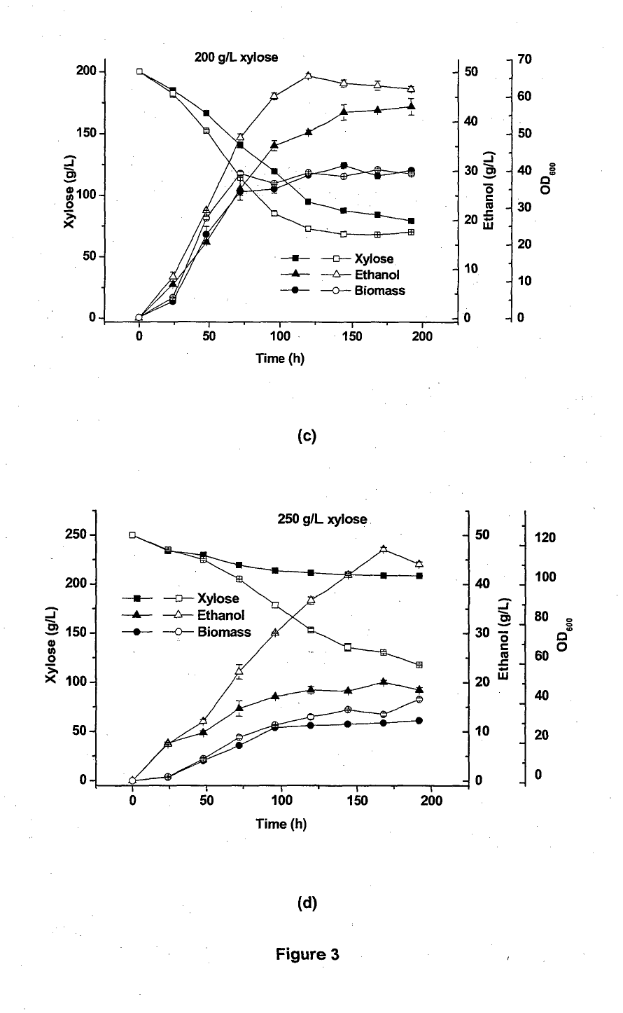

In one embodiment, there is provided a method of fermenting sugar as described in the Experimental Section. Examples of the result of the fermentation method are FIGS. 3, 4, 5, 6, 7 and 8.

It is to be understood that this invention is not limited to particular systems, devices or biological systems, which can, of course, vary. It is also to be understood that the terminology used herein is for the purpose of describing particular embodiments only, and is not intended to be limiting. As used in this specification and the appended claims, the singular forms “a”, “an” and “the” optionally include plural referents unless the content clearly dictates otherwise. Thus, for example, reference to “a yeast cell” includes a combination of two or more cells (e.g., in a culture); reference to “bacteria” includes mixtures of bacteria, and the like.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Unless specified otherwise, the terms “comprising” and “comprise”, and grammatical variants thereof, are intended to represent “open” or “inclusive” language such that they include recited elements but also permit inclusion of additional, unrecited elements.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Methods

### Strains and Media

Pichia stipitis CBS 6054, a haploidy yeast, was obtained from Centraalbureau voor Schimmelcultures (CBS, Baarn) Culture Collection, and it was maintained on YPX agar slants containing (g/L): xylose, 20.0; yeast extract, 10.0; peptone, 20.0; agar, 20.0 at pH 5.5±0.2. Saccharomyces cerevisiae ATCC 24860, a diploidy yeast, was procured from American Type Culture Collection (ATCC) and it was maintained on YPD agar slants containing (g/L): glucose, 20.0; yeast extract, 10.0; peptone, 20.0; agar, 20.0 at pH 5.5±0.2. They were stored in YPX or YPD broth containing 20% glycerol at −80° C. and were subcultured on YPX and YPD plates, respectively, at regular intervals. Yeast cells from freshly streaked YPD plates were inoculated in YPD broth and incubated at 30° C. and 200 rpm for 24 h. Cells were harvested and used as the source for genomic DNA extraction, direct genome transformation or as the inoculum for fermentation experiments.

### Genomic DNA Extraction

Cells of Pichia stipitis CBS 6054 were cultured in 50-mL centrifuge tubes containing 10 mL YPD broth at 30° C. and 200 rpm overnight. They were harvested after centrifugation at 5000×g at 4° C. for 5 min and then were washed with 20 mL sterile water three times. Cells were resuspended in 200 μL lysis buffer (100 mM Tris-HCl pH8.0, 50 mM EDTA and 0.5% SDS) and were transferred to a 1.5 mL microcentrifuge tube. Then 0.2 g glass beads (0.5 mm) were added to resuspend the cells. Cell suspension was thoroughly mixed at the maximal speed on a high speed vortex mixer. After centrifugation at 5000×g for 5 min at 4° C., the supernatant was transferred to a new 1.5 mL microcentrifuge tube and 500 μL phenol:chloroform:isoamyl alcohol (25:24:1) was added to the supernatant. This mixture was then briefly mixed on the vortex mixer and was centrifuged again at 12000×g and 4° C. for 10 min. The upper layer was then withdrawn carefully and was transferred to a new 1.5 mL microcentrifuge tube. One mL ice-cold 95% (v/v) ethanol was added to the supernatant and was briefly mixed by inversion. It was then stored at −20° C. for 2 h to precipitate the genomic DNA. After that, the sample was centrifuged at 12000×g and 4° C. for 10 min and the supernatant was carefully discarded to retain the genome DNA pellet. Afterwards, 1 mL 75% (v/v) ice-cold ethanol was used to wash the genomic DNA pellet three times and the DNA pellets were then dried by incubation at 37° C. for 1 h. The genomic DNA was resuspended in 200 μL of sterile water and was stored at −20° C. until use.

### Electroporation

The host yeast strain S. cerevisiae was cultured in 150-mL shaking flasks containing 50 mL YPD broth at 30° C. and 200 rpm overnight. Cells were harvested by centrifugation at 5000×g and 4° C. for 5 min and were washed three times with 20 mL sterile water each time. Cells were resuspended in 20 mL pretreatment-solution (0.1 M lithium acetate, 0.1 M Dithiothreitol (DTT), 0.6 M sorbitol, 0.01 M Tris-HCL of pH7.5) and incubated at room temperature for 30 min. The solution was centrifuged at 5000×g and 4° C. for 5 min and the supernatant was discarded. Cells were then resuspended in 20 mL 1 M sorbitol and centrifuged again under the same conditions. Again, the supernatant was discarded. Cells were then resuspended in 80 μL 1 M sorbitol solution and mixed with 20 μL of the isolated P. stipitis genomic DNA solution. The mixed solution was transferred into an electroporation cuvette and incubated in ice for about 5 min. Electroporation was then conducted using Gene Pulser Xcell™ electroporation system (Bio-Rad, USA) under the prescribed conditions according to the manufacturer's instructions. After electroporation, 1 mL 1 M sorbitol solution was added into the cuvette gently. The cuvette was then incubated at 30° C. for about 2 h. The transformed cells were then resuspended in 50 mL sterile centrifuge tube containing 5 mL YPD broth and incubated at 30° C. and 200 rpm for 3 h. The cultivation broth was spread on the predefined screening plates. Afterwards, the plates were incubated at 30° C. for 7-10 days. Positive clones were then selected, subcultured on YPD plates and were evaluated in shaking flasks for xylose fermentation. Potential recombinant strains will be used as the host for the next round of whole genome transformation.

### Random Amplified Polymorphic DNA (RAPD)

The RAPD reactions were performed using decamer primers of the OPERON random primer kit (OPA 01, 02, 03, 07, 08, 09 and 10), and the arbitrary primers SOY, RP1-4, RP-2, RP4-2 listed in Table 2. The amplification were conducted with a predenaturation at 94° C. for 10 min followed by 44 cycles of thermal denaturation at 94° C. (45 sec), primer annealing at 36° C. (45 sec), and extension at 72° C. (2 min). After that, a 10 min final extension at 72° C. was conducted to stabilize the amplified DNA products. Such amplified products were separated by electrophoresis in 1.0% agarose gel, 1×TAE buffer (40 mM Trisbase-Acetate and 1 mM Na₂EDTA, pH8.0) and a constant voltage of 120 V, using a horizontal electrophoresis (Cleaver, UK) followed by staining with SYBR Safe (ABM) and visualization in a UV transilluminator.

### Shaking Flask Fermentation

One loop of the positive clones was transferred from 1-day YPD plates to 150-mL Erlenmeyer flask containing 50 mL of YPD broth. Yeasts were grown for 24 h at 200 rpm on a rotary shaker at 30° C. A small volume of such seed culture was inoculated to each 150-mL Erlenmeyer flask containing 50 mL of the fermentation medium (FM) containing (g/L) yeast extract, 7; Peptone, 2; (NH₄)₂SO₄, 2; KH₂PO₄, 2.05; Na₂HPO₄, 0.25 to make an initial inoculum size of 0.5 OD₆₀₀. The Erlenmeyer flasks were shaken at 100 rpm and 30° C. Samples were withdrawn periodically to determine the concentration of sugar, ethanol, xylitol and cell biomass. Fermentation experiments were conducted in duplicate.

### Analytical Methods

Cell biomass was monitored spectrophotometrically by measuring absorbance at 600 nm. The measurement was made such that the optical density (OD₆₀₀) of the samples was smaller than 0.70, as obtained by sample dilution. This is to ensure that the Beer-Lambert law applies. Samples were filtered through 0.45 μm filters and stored at −20° C. until analysed by a 1200 Series HPLC system (Agilent Technologies Inc.) equipped with a Refractive Index Detector. Sugars, ethanol and xylitol were analysed on a Sugar-Pak.column (Waters, USA) at 75° C. with the mobile phase of 0.001 mM EDTA-Ca and a flow rate of 0.4 mL/min.

### Sugar Utilization Tests

Sugar utilization tests were carried out in YNB broth containing 6.7 g/L yeast nitrogen base (YNB) and 2 g/L of various tested sugars individually. ScF2 and its parents (P. stipitis and S. cerevisiae) were inoculated into 50 mL centrifuge tubes containing 10 mL YNB broth with each tested sugar. YNB broth without sugar was used as the control. These tubes were incubated in an orbital shaker at 200 rpm and 30° C. for 48 h and experiments were conducted in duplicate. At the end of the experiments, OD₆₀₀ was measured and compared.

### Simultaneous Saccharification and Fermentation of Oil Palm Empty Fruit Bunch Hydrolylate

Oil palm empty fruit bunch (OPEFB) was obtained from an oil palm plantation in Malaysia. OPEFB was washed, stored and milled as described in Wang et al. (Wang Z S, Ong H X and Geng A L*. Cellulase production and oil palm empty fruit bunch saccharification by a new isolate of Trichoderma koningii D-64, Process Biochemistry, 2012, 47: 1564-1571, DOI: 10.1016/j.procbio.2012.07.001.) Ten per cent (w/v) of the milled biomass particles (<500 μm) was pretreated by chemically using alkali and hydrogen peroxide. The pretreated OPEFB contained 53.2% cellulose, 21.9% xylose and 13.1% lignin. The rest are extractives and ash. Ten percent (w/w) of the pretreated OPEFB was loaded in the 250 mL conical flasks for a pre-hydrolysis using commercial enzymes Celluclase 1.5 L and Novozyme 188 in 50 mM citrate buffer (pH 4.8) with the FPase to beta-glucosidase ratio of 1:3 and FPase loading of 30 FPU/g biomass at 30° C. in an orbital shaker at 150 rpm for 3 h. The total liquid volume was 50 mL. ScF2 was precultured at the given conditions described in previous sections and washed using sterile distilled water. Approximately, one gram (dry weight) of the washed ScF2 was then supplemented to the 3-h hydrolysate to start the simultaneous saccharfication and fermentation (SSF) under the same conditions. The 3-h prehydrolysis sample without inoculating of the yeast was used as the control for continual hydrolysis. Sugar and product content in the fermentation broth was analysed by high performance liquid chromatography (HPLC) in an Agilent HPLC system with refractive index detector. HPLC organic acid analysis column (Aminex HPX87H ion exclusion column, 300 mm×7.8 mm. Bio-Rad) was equilibrated at 55° C. using the mobile phase of 5 mM H₂SO₄ at a flow rate of 0.6 mL/min.

### Results

### Modified Method of Genome Shuffling

In this present disclosure, S. cerevisiae and P. stipitis were used as the parents for recombinant yeast strain construction. In the first round, the whole genome of P. stipitis was extracted and transferred into S. cerevisiae by electroporation. The recombinant strains were selected on YNBX plates containing 6.7 g/L yeast nitrogen base, 50 g/L xylose and 20 g/L agar. Such plates were incubated at 30° C. for 7-10 days. S. cerevisiae cannot grow under the same conditions. Eight hybrid yeast strains were obtained and they were further evaluated for ethanol production in YNB broth containing 6.7 g/L YNB, 150 g/L xylose, and 50 mM phosphate buffer at pH 7.0 and 30° C. for 72 h. The potential recombinant strain with the best ethanol production performance was F1-8 (Table 1). This strain was then used as the starting strain for the second round of genome shuffling.

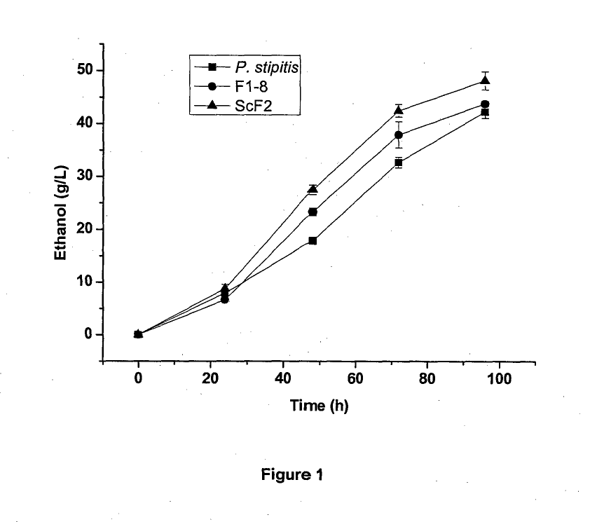

In the second round, the whole genome of S. cerevisiae was transferred into F1-8 by electroporation and the recombinant strain was screened on YNBXE plates containing 6.7 g/L yeast nitrogen base, 50 g/L xylose, 50 g/L ethanol and 20 g/L agar. Hybrid yeast strain F1-8 showed no growth on this selective plate. Three positive colonies were obtained and the strain with the most potential was ScF2 according to their competency in ethanol production. As a reference, protoplast fusion was conducted to construct the hybrid yeast using F1-8 and S. cerevisiae. None of the fusants survived on the same YNBXE selective plates. Afterwards, the xylose fermentation capability of the potential recombinant strains F1-8 and ScF2, and their parents, P. stipitis, were evaluated in 150 mL shaking flasks filled with 50 mL of the fermentation medium containing 120 g/L xylose. The results are shown in FIG. 1. As can be seen, ScF2 presented improved ethanol production rate and ethanol titre compared to both P. stipitis and F1-8.

### Random Amplified Polymorphic DNA (RAPD)

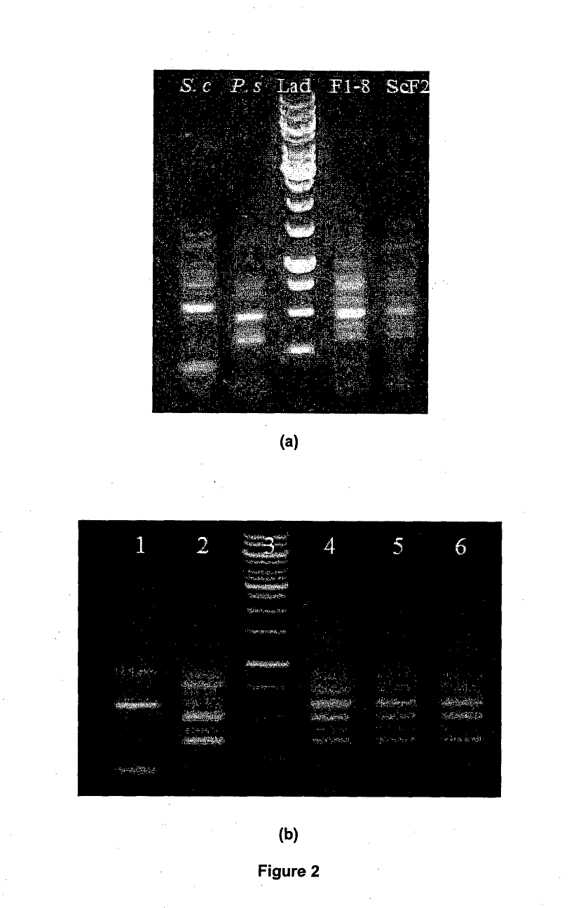

To obtain molecular evidence of the occurrence of recombinatory events using the modified genome shuffling method, the amplification profiles of parental strains and the potential recombinant strains were compared by random amplified polymorphic DNA analysis (RAPD). Using OPA kit, RP1-4, RP-2, RP4-2 and SOY as primers (Table 2), a large number of DNA bands were obtained from the templates of the recombinant yeast strain genomes (FIG. 2). Differences were clearly observed between the RAPD profiles of the parents and ScF2 (FIG. 2a). Consistent RAPD profiles were seen with ScF2 obtained at different time points over a period of nine months (FIG. 2b).

### Sugar Utilization

The hybrid nature of ScF2 was confirmed by comparing its sugar utilization pattern with those of its two parental strains (Table 3). Combined sugar utilization characteristics of S. cerevisiae and P. stipitis were observed for the recombinant strain ScF2. ScF2 demonstrated enhanced performance for fructose, xylose, maltose and cellobiose compared to both of the parental strains. It displayed decreased glucose and raffinose utilization capability than S. cerevisiae, and less mannose, sucrose and lactose utilization than P. stipitis. It showed a similar sugar utilization pattern with P. stipitis for the rest of the sugars listed in Table 3.

### Fermentation Performance of ScF2 in High Initial Xylose Concentration

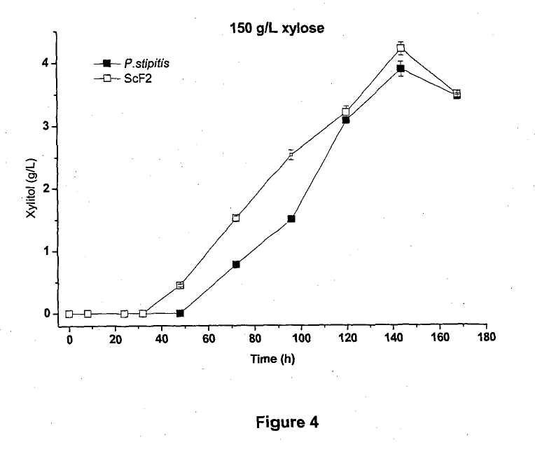

Xylose fermentation that was conducted in high initial xylose concentrations (100, 150, 200, and 250 g/L) using ScF2 and P. stipitis was studied. The results are shown in FIG. 3. At initial concentration of 100 g/L, xylose was completely utilized on day 3 by both strains and 42 g/L of ethanol was obtained by ScF2 and 38 g/L by P. stipitis. The maximum ethanol production of 51 g/L was obtained on day 5 in 150 g/L xylose by ScF2, whereas 48 g/L ethanol was obtained by P. stipitis under the same conditions. In addition, recombinant strain ScF2 demonstrated slightly higher rates of xylose consumption and ethanol production in both of the above initial xylose concentration. When the initial xylose concentration was increased further to 200 g/L, the difference between the rates of xylose consumption and ethanol production by ScF2 and P. stipitis became more noticeable. Approximately 49 g/L ethanol was obtained by ScF2 on day 5, whereas 43 g/L ethanol was obtained by P. stipitis on day 8. At initial xylose concentration of 250 g/L, xylose consumption and ethanol production by P. stipitis were significantly inhibited by the high content of xylose and about 20 g/L of ethanol was obtained on day 7. On the other hand, the high xylose content only slightly inhibited xylose consumption and ethanol production by ScF2 with the maximal ethanol concentration of 47 g/L on day 6. The highest ethanol titre of 51 g/L was obtained by the recombinant strain ScF2 in 150 g/L initial xylose concentration. Further increase of the initial xylose concentration triggered a slight decrease in the maximal ethanol titre and an increase of the fermentation time. Although ScF2 demonstrated much higher xylose tolerance and improved ethanol titre compared to P. stipitis, its ethanol titre was only limited to around 50 g/L due to the incomplete conversion of xylose. Similar to its parent, P. stipitis, the main byproduct for the recombinant strain ScF2 was xylitol. With the enhancement of ethanol production, its xylitol production rate was also higher than that of P. stipitis (FIG. 4).

Fermentation of Glucose, Xylose and their Mixture

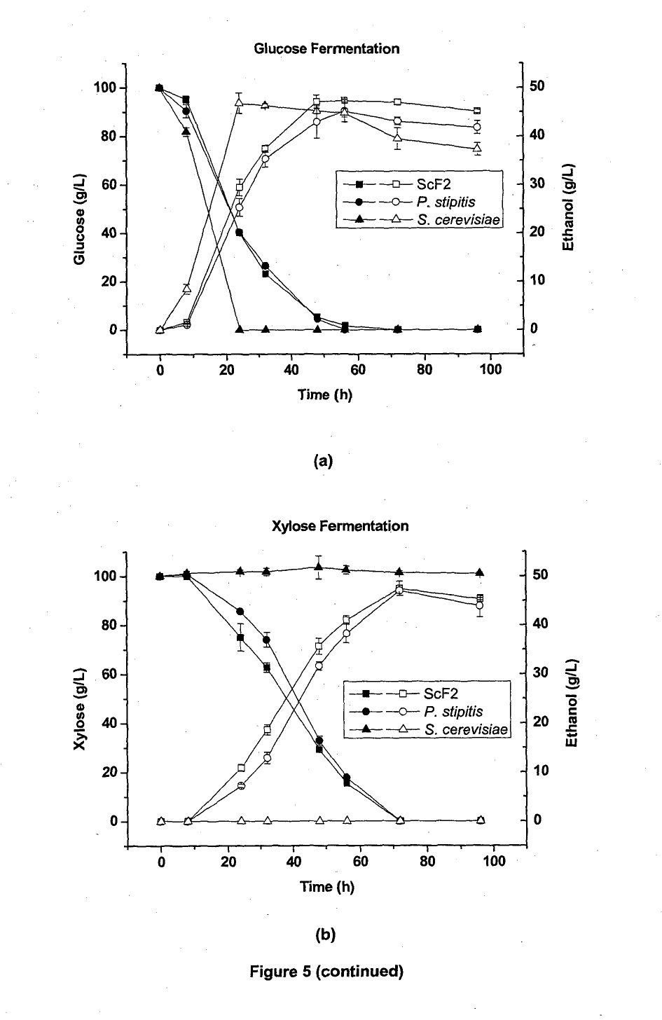

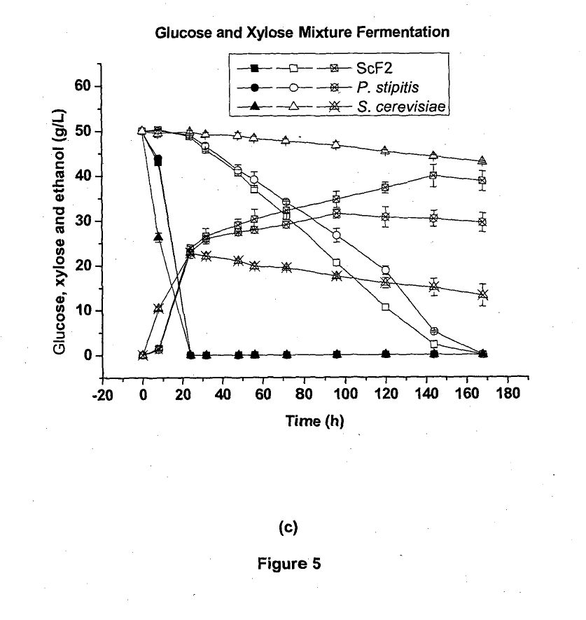

In this part of the study, the fermentation of glucose, xylose and their mixture by strains P. stipitis, S. cerevisiae and ScF2 were investigated independently under batch cultivation conditions. The total sugar concentration was maintained at 100 g/L for all experiments and experiments were conducted in duplicate. As shown in FIG. 5, P. stipitis and ScF2 could utilize both glucose and xylose, while S. cerevisiae could only utilize glucose. Glucose was completely consumed by S. cerevisiae within 24 h, by P. stipitis within 48 h, and by ScF2 in 56 h. However, ScF2 produced more ethanol (47 g/L) than P. stipitis (45 g/L) from glucose. Complete utilization of xylose was observed for both ScF2 and P. stipitis, with the former being faster in the rates of both xylose consumption and ethanol production. For the case of glucose and xylose mixture fermentation, again ScF2 and P. stipitis could utilize both sugars, with glucose being consumed in a much faster rate. S. cerevisiae strain only consumed glucose and the maximal ethanol concentration was 22 g/L. Slight decrease of xylose consumption rate was also observed for both ScF2 and P. stipitis under this condition compared to the case when xylose was used as the sole carbon source. In addition, ScF2 exhibited slightly higher rates for both xylose consumption and ethanol production than P. stipitis. The maximal ethanol concentration of 40 g/L was obtained for ScF2 at 144 h, and that for P. stipitis was 31 g/L at 96 h.

### Xylose Fermentation by ScF2 Precultured in High-Concentration Glucose or Xylose

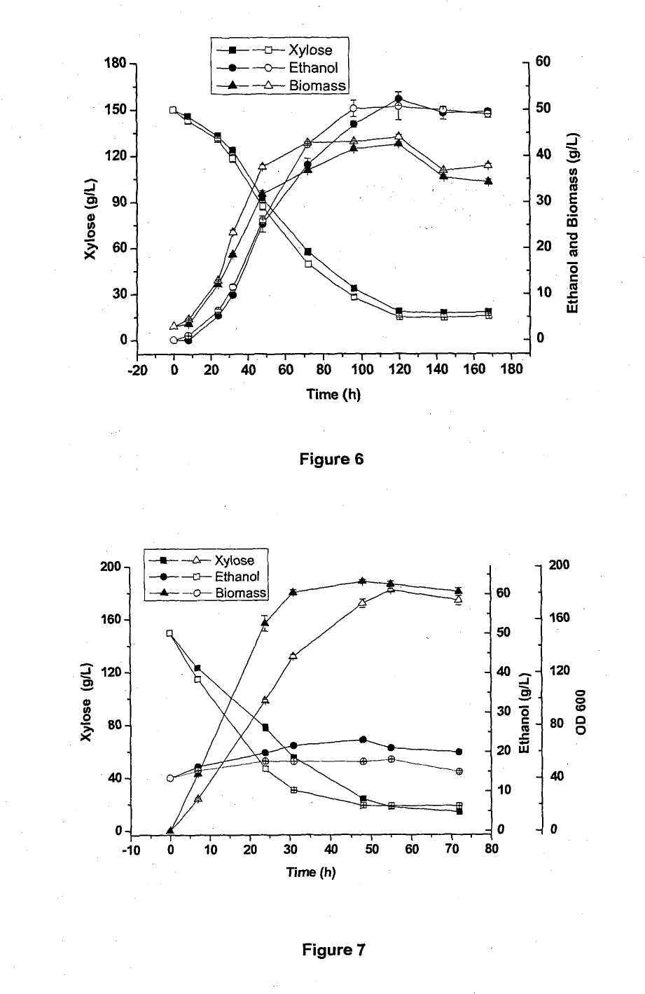

It was reported that metabolic lag existed for substrate transition. This indicates that yeast strain precultured on glucose prior to its use as inoculum for xylose fermentation may lead to longer metabolic lag phase. In order to further improve xylose fermentation performance by ScF2, seeds culture of ScF2 was prepared in yeast peptone medium containing 10 g/L yeast extract, 20 g/L peptone, and 150 g/L glucose or xylose. Cells were harvested and inoculated to fresh fermentation medium containing 150 g/L xylose at an initial OD₆₀₀ of 3.0. Results are displayed in FIG. 6. Slight enhancement of cell growth and ethanol production by ScF2 precultured in xylose were observed. The maximal ethanol titre was obtained at 96 h by xylose precultured ScF2 and at 120 h by glucose precultured ScF2 (Table 4). Interestingly, although preculture in glucose resulted in a slightly longer lag phase for cell growth and ethanol production, a marginally higher ethanol titre, 52 g/L, was obtained compared with the preculture in xylose (Table 4). Noticeably, despite the difference in preculture substrates, ScF2 presented higher xylose consumption rate and ethanol productivity compared to P. stipitis. This concurred with the results obtained in previous sections.

### High Density ScF2 Xylose Fermentation

In previous sections, the initial cell density was controlled as 3.0 OD₆₀₀. It took 48-74 hours for biomass density to reach its maximum (FIGS. 3 and 6). High cell density is favorable for both ethanol production rate and ethanol yield. In order to further improve xylose fermentation performance by ScF2, seeds culture of ScF2 was prepared in yeast peptone medium containing 10 g/L yeast extract, 20 g/L peptone, and 150 g/L glucose or xylose. Cells were harvested and inoculated to fresh fermentation medium containing 150 g/L xylose at an initial OD₆₀₀ of 40.0. Results are displayed in FIG. 7. ScF2 was seen to display much faster rates for ethanol production and xylose consumption. The highest ethanol titer reached 63 g/L at 48 h, the ethanol productivity reached 2.0 g/L/h at 30 h with an ethanol concentration of 60 g/L and yield of 0.40 g/g. In contrast, it took 55 h for Pichia stipitis to reach the highest ethanol concentration of 60 g/L, corresponding to a productivity of 1.09 g/L/h, which was much slower than ScF2. These results suggest that high cell density is useful in enhancing ethanol productivity, concurring with the previous reports.

### Simultaneous Saccharification and Fermentation of Oil Palm Empty Fruit Bunch Hydrolylate

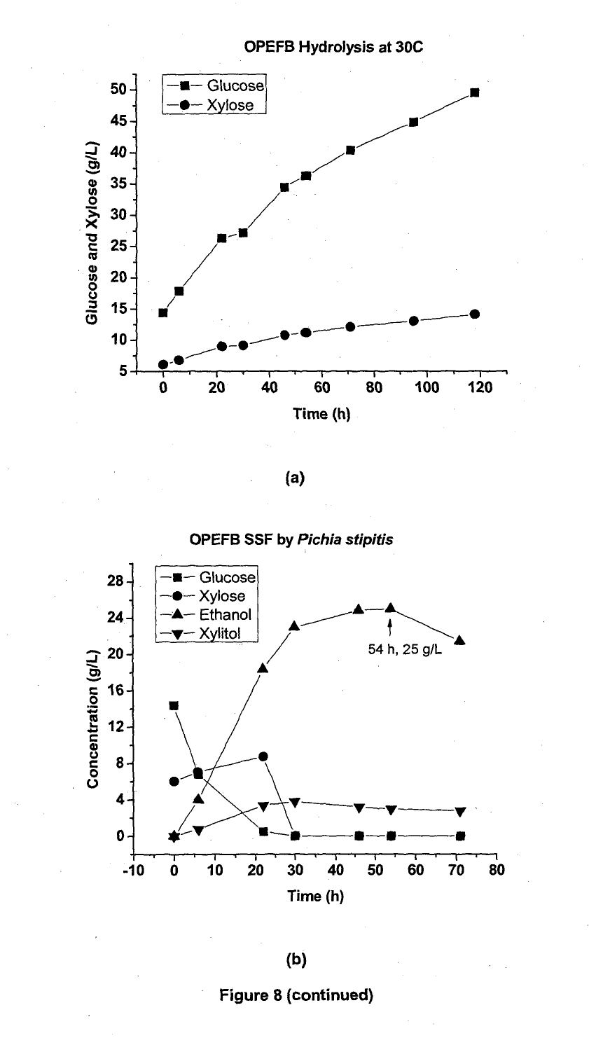

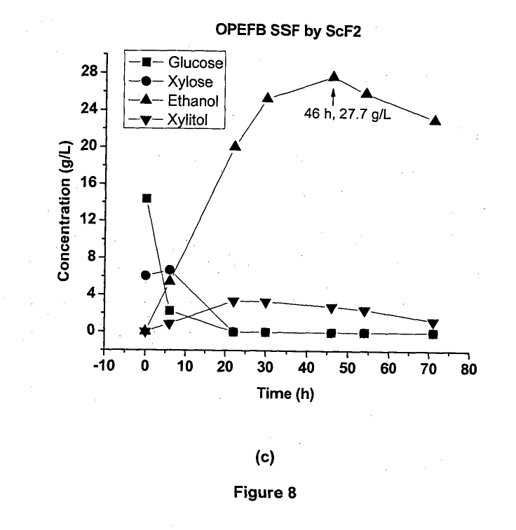

Crude palm oil production reached 48.99 million metric tonnes per year globally in 2011 and Southeast Asia is the main contributor, with Indonesia accounting for 48.79%, Malaysia 36.75%, and Thailand 2.96% (Palm Oil Refiners Association of Malaysia, 2011). Oil palm biomass, including oil palm empty fruit bunch (OPEFB), oil palm trunks and fronds, is therefore one of the biomass resources with the most potential that is available in Southeast Asia. Among them, OPEFB is the most abundant and digestible. OPEFB was used in this study for cellulosic ethanol production using strain ScF2 through simultaneous saccharification and fermentation (SSF) at 30° C. Although the ideal temperature for OPEFB hydrolysis is 50° C., that for yeast alcoholic fermentation is 30° C. Therefore, SSF experiments were conducted at 30° C. FIG. 8 (a) displays the hydrolysis results at this temperature and about 63.5 g/L sugar was released after 118 h hydrolysis, whereas about 80 g/L of sugar can be produced at 50° C. In SSF of OPEFB using Pichia stipitis, the highest ethanol concentration was 25 g/L at 54 h (FIG. 8 (b)), while 27.7 g/L of ethanol was produced at 46 h using strain ScF2 (FIG. 8(c)). The ethanol yield was 0.44 g/g based on the highest amount of sugar obtained at 118 h of hydrolysis at 30° C. (FIG. 8(a)). SSF is very advantageous in the enhancement of hydrolysis rate as the time to release the same amount of sugar was decreased from 118 h to 46 h for ScF2 and to 54 h for P. stipitis. No further ethanol increase was observed after that due to the inability of further hydrolysis at 30° C. (FIG. 8 (a)). Both ethanol titre and ethanol productivity was improved using ScF2. Strain ScF2 is therefore a potential strain that can be used for cellulosic ethanol production through SSF.

Disclosed herein is one example of a modified genome shuffling method for rapid construction of a recombinant yeast strain from S. cerevisiae and P. stipitis. In combination with properly designed screening strategy, a potential hybrid yeast ScF2 was constructed. This hybrid yeast displayed improved tolerance to xylose and ethanol, and enhanced rates of xylose consumption and ethanol production compared to their parents. Combined with proper screening strategy, the modified genome shuffling method was effective and easy to operate for the construction a recombinant strain with desired phenotypes in a short time.

### Tables

TABLE 1
Fermentation performance of first round hybrid yeasts in YNBX broth
containing 150 g/L xylose.

| | P. | | | | | | | | |
|---|---|---|---|---|---|---|---|---|---|
| | stipitis | F1-1 | F1-2 | F1-3 | F1-4 | F1-5 | F1-6 | F1-7 | F1-8 |
| Ethanol | 0.27 ± 0.01 | 0.28 ± 0.01 | 0.29 ± 0.03 | 0.28 ± 0.02 | 0.29 ± 0.02 | 0.29 ± 0.01 | 0.29 ± 0.01 | 0.29 ± 0.02 | 0.31 ± 0.03 |
| yield | | | | | | | | | |
| (g/g) | | | | | | | | | |
| Ethanol | 0.32 ± 0.01 | 0.33 ± 0.01 | 0.35 ± 0.01 | 0.34 ± 0.01 | 0.35 ± 0.04 | 0.36 ± 0.02 | 0.35 ± 0.01 | 0.36 ± 0.01 | 0.38 ± 0.02 |
| produc- | | | | | | | | | |
| tivity | | | | | | | | | |
| (g/L/h) | | | | | | | | | |

TABLE 2
Primers used for random amplified polymorphic DNA.

| Primers-Operon/ | 10-mer in length- | SEQ |
|---|---|---|
| Design | 5′ to 3′ | ID NO: |
| OPA01 | CAGGCCCTTC | 1 |
| OPA02 | TGCCGCGCTG | 2 |
| OPA03 | AGTCAGCCAC | 3 |
| OPA07 | GAAACGGGTG | 4 |
| OPA08 | GTGACGTAGG | 5 |
| OPA09 | GGGTAACGCC | 6 |
| OPA10 | GTGATCGCAG | 7 |
| RP1-4 | TAGGATCAGA | 8 |
| RP2 | AAGGATCAGA | 9 |
| RP4-2 | CACATGCTTC | 10 |
| SOY | AGGTCACTGA | 11 |

Unless otherwise indicated, nucleic acids are written left to right in 5′ to 3′ orientation; amino acid sequences are written left to right in amino to carboxy orientation, respectively.

TABLE 3
Sugar utilization by ScF2 and its parental strains.

| | S. cerevisiae | ScF2 | P. stipitis |
|---|---|---|---|
| control | − | − | − |
| Hexoses | | | |
| glucose | +++ | ++ | ++ |
| fructose | + | +++ | ++ |
| galactose | ± | + | + |
| raffinose | ± | − | − |
| mannose | + | + | ++ |
| rhamnose | ± | + | + |
| Pentose | | | |
| xylose | − | +++ | ++ |
| L-arabinose | − | ± | ± |
| D-arabinose | − | − | − |
| ribose | − | ± | ± |
| Disaccharides | | | |
| sucrose | + | + | ++ |
| lactose | − | − | ± |
| cellobiose | − | +++ | ++ |
| Maltose | + | ++ | + |

−, not growth;
±, feeble growth;
+, slow growth;
++, moderate growth;
+++, fast growth

TABLE 4
Xylose fermentation parameters with ScF2 inoculum pre-cultured in 150 g/L
glucose or xylose.

| | | Ethanol | | Xylose | Ethanol | |
|---|---|---|---|---|---|---|
| | | (g/L) | Yield (g/g) | (g/L/h) | (g/L/h) | Time (h) |
| ScF2 | Pre-G | 52.25 ± 1.48 | 0.40 ± 0.01 | 1.10 ± 0.00 | 0.44 ± 0.01 | 120 |
| | Pre-X | 50.20 ± 1.78 | 0.37 ± 0.01 | 1.28 ± 0.01 | 0.52 ± 0.02 | 96 |
| P stipitis | Pre-G | 52.15 ± 0.28 | 0.38 ± 0.00 | 0.95 ± 0.01 | 0.36 ± 0.00 | 144 |
| | Pre-X | 49.94 ± 0.62 | 0.37 ± 0.01 | 0.93 ± 0.01 | 0.35 ± 0.00 | 144 |

### Application

It will be apparent that various other modifications and adaptations of the invention will be apparent to the person skilled in the art after reading the foregoing disclosure without departing from the scope of the invention and it is intended that all such modifications and adaptations come within the scope of the appended claims.

The present disclosure provides for a rapid and reliable method for constructing a recombinant yeast strain.

The present disclosure provides for a method of constructing recombinant yeast strain that has enhanced xylose-fermentation.

The method as disclosed herein advantageously provides for rapid complex phenotype improvement as seen in protoplast fusion-based genome shuffling method.

The method as disclosed herein is also time-saving, easier to operate and has higher gene recombination efficiency.

The method as disclosed herein advantageously provides for a useful method for developing recombinant microorganisms that are capable of producing biofuels (such as bioethanol, biobutanol, carbohydrates and the likes), biobased chemicals (such as organic acids, polymers, materials) and fine chemicals (such as fragrants, nutraceutical and pharmaceutical products etc).

The recombinant yeast as obtained from the method of the present disclosure advantageously has enhanced xylose-fermentation and has higher ethanol tolerance as compared to its parents strains.

The recombinant yeast as obtained from the method of the present disclosure allows for efficient production of biofuel, such as bioethanol, from lignocellulosic biomass.

1. A method of providing a recombinant microorganism, said method comprising:
(a) providing a hybrid microorganism comprising whole genomic DNA from a host microorganism and a donor microorganism, wherein the hybrid microorganism is thus a microorganism having genomes from two different parent microorganism, the hybrid microorganism is produced by transferring the whole genome of the donor microorganism to the host microorganism by whole genome transformation; and the hybrid microorganism exhibits an enhanced activity as compared to its host microorganism and/or donor microorganism individually; and
(b) fusing whole genomic DNA extracted from a second microorganism into the hybrid microorganism to form said recombinant microorganism.

2. The method of claim 1, wherein said second microorganism of step (b) is of the same species as the host or donor microorganism in (a).

3. (canceled)

4. The method of claim 1, further comprising:
(c) screening said recombinant microorganism and selecting a recombinant strain expressing two or more desired traits; and
(d) fusing DNA extracted from a microorganism expressing at least one of said desired traits into said selected recombinant strain to obtain further recombinant microorganisms.

5. The method of claim 4, further comprising repeating (c) and (d) until said desired traits are expressed in the recombinant microorganisms.

6. The method of claim 1, wherein said microorganism is selected from the group consisting of bacteria, fungi, and yeast.

7. (canceled)

8. The method according to claim 6, wherein at least one of said host or donor microorganism is a yeast cell capable of fermenting xylose.

9. The method according to claim 6, wherein at least one of the said host or donor yeast cell is tolerant to ethanol.

10. The method according to claim 6, wherein the hybrid yeast cell is obtained by screening a plurality of hybrid yeast cell lines for capability to ferment xylose.

11. The method according to claim 10, wherein said plurality of hybrid yeast cell lines are provided by mixing a suspension of said host yeast cells with extracted DNA from said donor yeast cells to achieve transfection of the extracted DNA into said host yeast cell.

12. The method according to claim 11, wherein said transfection is carried out using a transfection method selected from the group consisting of chemical based transfection, non-chemical based transfection, particle-based transfection and viral methods.

13. The method of claim 12, wherein the chemical based method is using calcium phosphate or dendrimers or liposomes or cationic polymers; or
the non-chemical transfection method is electroporation or sono-poration or optical transfection or gene electrotransfer or hydrodynamic delivery; or
the particle-based transfection is using a gene gun or magnetofection or impalefection.

14. (canceled)

15. (canceled)

16. The method according to claim 6, wherein said host and donor yeast cells belong to different or the same taxonomic family.

17. The method according to claim 16, wherein said host and donor yeast cells belong to the family Saccharomycetaceae.

18. The method according to claim 17, wherein said donor yeast cell is of a genus selected from the group consisting of: Brettanomyces, Candida, Citeromyces, Cyniclomyces, Debaryomyces, Issatchenkia, Kazachstania, Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, and Zygotorulaspora.

19. (canceled)

20. The method according to claim 18, wherein said donor yeast cell is of a species selected from the group consisting of: Pichia pastoris, Pichia guilliermondii, Pichia membranifaciens, Pichia heedii, Pichia stipitis, and Pichia subpelliculosa.

21. (canceled)

22. The method according to claim 17, wherein said host yeast cell is of a genus selected from the group consisting of: Brettanomyces, Candida, Citeromyces, Cyniclomyces, Debaryomyces, Issatchenkia, Kazachstania, Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, and Zygotorulaspora.

23. (canceled)

24. The method according to claim 22, wherein said host yeast cell is of a species selected from the group consisting of: Saccharomyces cerevisiae, Saccharomyces pastorianus, Saccharomyces boulardii, Saccharomyces eubayanus, Saccharomyces bayanus, Saccharomyces bailii, and Saccharomyces florentinus.

25. (canceled)

26. A recombinant yeast strain produced according to a method of providing a recombinant microorganism, said method comprising:
(a) providing a hybrid microorganism comprising whole genomic DNA from a host microorganism and a donor microorganism, wherein the hybrid microorganism is thus a microorganism having genomes from two different parent microorganism, the hybrid microorganism is produced by transferring the whole genome of the donor microorganism to the host microorganism by whole genome transformation; and the hybrid microorganism exhibits an enhanced activity as compared to its host microorganism and/or donor microorganism individually; and
(b) fusing whole genomic DNA extracted from a second microorganism into the hybrid microorganism to form said recombinant microorganism.

27. A method of fermenting sugar with a recombinant yeast strain to produce ethanol, wherein the recombinant yeast is produced according to a method of providing a recombinant microorganism, said method comprising:
(a) providing a hybrid microorganism comprising whole genomic DNA from a host microorganism and a donor microorganism, wherein the hybrid microorganism is thus a microorganism having genomes from two different parent microorganism, the hybrid microorganism is produced by transferring the whole genome of the donor microorganism to the host microorganism by whole genome transformation; and the hybrid microorganism exhibits an enhanced activity as compared to its host microorganism and/or donor microorganism individually; and
(b) fusing whole genomic DNA extracted from a second microorganism into the hybrid microorganism to form said recombinant microorganism.

28. The method according to claim 27, wherein said sugar is selected from the group consisting of hexoses, pentoses, disaccharides and mixtures thereof.

29. The method according to claim 28, wherein the hexoses are selected from the group consisting of glucose, fructose, galactose, mannose, rhamnose and mixtures thereof; or
the pentoses are selected from the group consisting of: xylose, L-arabinose, D-arabinose, ribose and mixtures thereof; or
the disaccharides are selected from the group consisting of: sucrose, lactose, cellobiose, maltose and mixtures thereof.

30. (canceled)

31. (canceled)

32. (canceled)

33. A method of fermenting a sugar mixture having a high sugar content with a recombinant yeast strain to produce ethanol, wherein the total sugar content is greater than 50 grams per liter, wherein the recombinant yeast strain is produced according to a method of providing a recombinant microorganism, said method comprising:
(a) providing a hybrid microorganism comprising whole genomic DNA from a host microorganism and a donor microorganism, wherein the hybrid microorganism is thus a microorganism having genomes from two different parent microorganism, the hybrid microorganism is produced by transferring the whole genome of the donor microorganism to the host microorganism by whole genome transformation; and the hybrid microorganism exhibits an enhanced activity as compared to its host microorganism and/or donor microorganism individually; and
(b) fusing whole genomic DNA extracted from a second microorganism into the hybrid microorganism to form said recombinant microorganism.

34. The method according to claim 33, wherein the total sugar content is from about 100 to about 300 grams per liter.

35. The method according to claim 33, wherein the sugar mixture is composed essentially of xylose.
